(19) United States
(12) Patent Application Publication
(10) Pub. No.: US 2026/0224464 A1
(43) Pub. Date: Aug. 6, 2026

(54) SKIN CARE COMPOSITION AND METHOD OF USING THE SAME

(71) Applicant: THE PROCTER & GAMBLE COMPANY, Cincinnati, OH (US)

(72) Inventors: Peter Brendan STYCZYNSKI, West Chester, OH (US); Chuiying LI, Mason, OH (US); Lu ZHANG, Singapore (SG)

(21) Appl. No.: 19/460,777

(22) Filed: Jan. 27, 2026

Related U.S. Application Data
(60) Provisional application No. 63/750,898, filed on Jan. 29, 2025.

(51) Int. Cl. A61K 8/64; A61K 8/67; A61Q 19/02

(52) U.S. Cl. CPC A61K 8/64; A61K 8/675; A61Q 19/02

(57) ABSTRACT

A skin care composition that includes a combination of peptides and a dermatologically acceptable carrier. The peptides include nicotinoyl tetrapeptide-30 (N-PKEK) [SEQ. ID NO: 1] and one or more peptides that are attached to a fatty acid moiety, such as palmitoyl moiety chosen from palmitoyl dipeptide-7 (pal-KT) and/or palmitoyl pentapeptide-4 (pal-KTTKS) [SEQ ID NO: 2]. The combination of peptides increases the total skin penetration and/or the epidermal skin penetration of N-PKEK [SEQ ID NO: 1].

### FIELD OF THE INVENTION

The present disclosure is directed generally to improving skin health and appearance by enhancing penetration of peptides. More specifically, the present disclosure is directed to use of one or more peptides that are attached to fatty acid moieties, such as palmitoyl including palmitoyl dipeptide-7 (pal-KT) and/or palmitoyl pentapeptide-4 (pal-KTTKS) [SEQ ID NO: 2], to enhance skin penetration of nicotinoyl tetrapeptide-30 (N-PKEK) [SEQ ID NO: 1].

### SEQUENCE LISTING

An official copy of the sequence listing is submitted concurrently with the specification electronically via EFS-Web as an ST.26 formatted sequence listing with a file name of “16862ε-Sequence.txt”, a creation date of Jan. 23, 2025, and a size of 952 bytes. The sequence listing contained in this ST.26 formatted document is part of the specification and is herein incorporated by reference in its entirety.

### BACKGROUND OF THE INVENTION

In recent years, consumers have become increasingly aware of skin health and aesthetics, increasing demand for effective skin care products. Many individuals, regardless of age or skin type, have hyperpigmentation and various types of spots, often referred to as age spots, sun spots, or dark spots, that can negatively impact a person's self-esteem and overall appearance. These irregularities are caused by various factors, including sun exposure, hormonal changes, aging, and environmental stressors.

Current topical skincare products often feature active ingredients, such as hydroquinone, kojic acid, and various alpha hydroxy acids (AHAs), all designed to diminish the visibility of skin discoloration. While some of these solutions can effectively lighten spots, they may come with risks including skin irritation, allergic reactions, or potential long-term damage with extended use. Additionally, many of the available products do not take a holistic approach, failing to address the root causes of hyperpigmentation and resulting in only temporary improvements.

Consequently, there is a need for topical skin care products that effectively target spots without causing irritation while also promoting skin health at a cellular level. Certain peptides have shown promise in reducing the appearance of spots and discoloration, with N-PKEK [SEQ ID NO: 1] and derivatives thereof emerging as a potential ingredient in formulations aimed at achieving a more even complexion. N-PKEK [SEQ ID NO: 1] derivatives can include acid, amide, ether, ester, amino, carboxyl, acetyl, alcohol derivative, or salts thereof, particularly the acid form (N-PKEK-acid, which has a —CO group attached to the lysine) and amide form (N-PKEK-amide, which features an —NH group attached to the lysine), or combinations thereof. However, its efficacy is limited due to poor skin penetration and bioavailability, stemming from its large molecular size and hydrophilic nature.

Therefore, there is also a need to identify compounds and methods that improve the efficacy of N-PKEK [SEQ ID NO: 1] in skin care compositions.

### SUMMARY OF THE INVENTION

A skin care composition comprising: (a) a combination of peptides comprising: (i) from about 1 ppm to about 50 ppm of a nicotinoyl tetrapeptide-30 (N-PKEK) [SEQ ID NO: 1], derivatives thereof, or mixtures thereof; and (ii) one or more peptides having a palmitoyl moiety chosen from palmitoyl dipeptide-7 (pal-KT) and/or palmitoyl pentapeptide-4 (pal-KTTKS) [SEQ ID NO: 2]; (b) a dermatologically acceptable carrier; wherein the combination increases the total skin penetration and/or the epidermal skin penetration of the N-PKEK [SEQ ID NO: 1].

A skin care composition comprising: (a) a combination of peptides comprising nicotinoyl tetrapeptide-30 (N-PKEK) [SEQ ID NO: 1], derivatives thereof, and mixtures thereof and one or more peptides that are attached to a fatty acid moiety; (b) a dermatologically acceptable carrier; wherein the composition increases the total skin penetration and/or the epidermal skin penetration of the N-PKEK [SEQ ID NO: 1].

### DETAILED DESCRIPTION OF THE INVENTION

Consumers want topical skin care products that effectively target spots without causing irritation while also promoting skin health at a cellular level. Some peptides have demonstrated potential in diminishing the appearance of spots and discoloration, with N-PKEK [SEQ ID NO: 1] identified as a promising ingredient for formulations designed to promote a more uniform complexion. However, N-PKEK [SEQ ID NO: 1] has limited effectiveness when applied topically because its relatively large molecular mass and hydrophilic nature prevent it from effectively penetrating the skin and becoming bioavailable.

It was surprisingly found that when N-PKEK [SEQ ID NO: 1] was used in combination with peptides that are attached to fatty acid moieties, such as palmitoyl including palmitoyl dipeptide-7 (pal-KT) and/or palmitoyl pentapeptide-4 (pal-KTTKS) [SEQ ID NO: 2], N-PKEK's [SEQ ID NO: 1] lipophilicity, skin penetration and bioavailability can be increased.

Table 1, below, used the Skin Penetration Test Method, described hereafter, to demonstrate the ability of pal-KT and pal-KTTKS [SEQ ID NO: 2] to enhance the skin penetration and bioavailability of N-PKEK [SEQ ID NO: 1].

Since N-PKEK [SEQ ID NO: 1] is an active ingredient that affects skin tone, it is important for it to penetrate to the epidermis, where melanocytes are generally found. Total penetration is also beneficial as N-PKEK [SEQ ID NO: 1] must first pass through the epidermis to reach the dermis or interact with receptors. As shown in Table 1, below, adding pal-KT and pal-KTTKS increased the penetration of the N-PKEK [SEQ ID NO: 1]. In the examples in Table 1, when the N-PKEK was added it was in the acid form, however, it is believed that the results would be similar if other derivatives of N-PKEK were added.

TABLE 1
N-PKEK [SEQ. ID. NO: 1] Skin Penetration

| | | | |
|---|---|---|---|
| Control | Ex. A | Ex. B | Ex. C |

Composition

| Chassis | Example 1 (see Table 2, hereafter) containing | | | |
|---|---|---|---|---|
| | 40 ppm N-PKEK [SEQ ID NO: 1] | | | |
| pal-KT | | 5 ppm | 10 ppm | 50 ppm |
| pal-KTTKS | | 3.5 ppm | 10 ppm | 50 ppm |
| [SEQ ID NO: 2] | | | | |

Skin Penetration Test Results

| | | | | |
|---|---|---|---|---|
| Skin Surface | 81.63% | 67.63% | 68.78% | 78.60% |
| Epidermis | 9.80% | 16.11% | 20.01% | 13.63% |
| Dermis | 6.34% | 6.66% | 7.45% | 5.04% |
| Receptor | 2.22% | 9.59% | 3.76% | 2.73% |
| Total Penetration | 18.37% | 32.37% | 31.22% | 21.40% |
| Fold Increase Epidermal | | 1.64 | 2.04* | 1.39* |
| N-PKEK Penetration | | | | |
| vs. Control | | | | |
| Fold Increase Total | | 1.76* | 1.70* | 1.17 |
| N-PKEK Penetration | | | | |
| vs. Control | | | | |

*Indicates the p-value is ≤0.05

The combination of N-PKEK [SEQ ID NO: 1] and one or more peptides that are attached to fatty acid moieties, such as palmitoyl including palmitoyl dipeptide-7 (pal-KT) and/or palmitoyl pentapeptide-4 (pal-KTTKS) [SEQ ID NO: 2] can be in a cosmetic product including a face wash, body wash, and/or leave-on skin care product such as a moisturizer including but not limited to creams, lotions, and gel creams and/or treatment products including but not limited to serums, toners, and essences. The skin care product can help provide spot fading, tone brightening, and/or tone evenness. The skin care product can also prevent pigmentation and/or hyper-pigmentation. The skin care product can help stabilize dendricity enabling melanin inhibition and provide other tone related benefits.

The skin care composition can include N-PKEK [SEQ ID NO: 1] and one or more peptides that are attached to fatty acid moieties and when the composition is tested according to the Skin Penetration Method, the N-PKEK can have a total skin penetration of ≥20, alternatively ≥21, alternatively ≥23, alternatively ≥25, alternatively ≥27, alternatively ≥29, alternatively ≥30, and alternatively ≥31.

The skin care composition can include N-PKEK [SEQ ID NO: 1] and one or more peptides that are attached to fatty acid moieties and when the composition is tested according to the Skin Penetration Method, the N-PKEK can have an epidermis penetration of ≥10, alternatively ≥12, alternatively ≥13, alternatively ≥15, alternatively ≥16, and alternatively ≥18.

The skin care composition can include N-PKEK [SEQ ID NO: 1] and one or more peptides that are attached to fatty acid moieties. The skin care composition can contain from about 5 to about 200 ppm of the one or more peptides one or more peptides that are attached to fatty acid moieties, alternatively from about 5 to about 150 ppm, alternatively from about 7 to about 100 ppm, alternatively from about 8 to about 50 ppm, alternatively from about 8 to about 30 ppm, alternatively from about 10 to about 30 ppm, and alternatively from about 15 to about 25 ppm.

When the composition is tested according to the Skin Penetration Method and compared to a control (which is identical except it lacks the one or more peptides that are attached to fatty acid moieties), the total skin penetration can have at least a 1.1-fold increase as compared to the control, alternatively at least a 1.15-fold increase, alternatively at least a 1.2-fold increase, alternatively at least a 1.3-fold increase, alternatively at least a 1.4-fold increase, alternatively at least a 1.5-fold increase, alternatively at least a 1.6-fold increase, alternatively at least a 1.65-fold increase, and alternatively at least a 1.7-fold increase.

The skin care composition can include N-PKEK [SEQ ID NO: 1] and one or more peptides that are attached to fatty acid moieties. When the composition is tested according to the Skin Penetration Method and compared to a control (which is identical except it lacks the one or more peptides that are attached to fatty acid moieties), the epidermal skin penetration can have at least a 1.2-fold increase as compared to the control, alternatively at least a 1.25-fold increase, alternatively at least a 1.3-fold increase, alternatively at least a 1.35-fold increase, alternatively at least a 1.38-fold increase, alternatively at least a 1.5-fold increase, alternatively at least a 1.6-fold increase, and alternatively at least a 1.8-fold increase.

### Tetrapeptide

The compositions herein include a safe and effective amount of the nicotinoyl tetrapeptide-30 (N-PKEK) [SEQ ID NO: 1] (INCI: nicotinoyl tetrapeptide-30) and derivatives thereof. The N-PKEK can be in acid and/or amide form and/or salts thereof. The N-PKEK [SEQ ID NO: 1] may be present at from about 0.01 ppm to about 100 ppm, alternatively from about 0.1 ppm to about 75 ppm, alternatively from about 1 ppm to about 50 ppm, alternatively from about 5 ppm to about 50 ppm, alternatively from about 10 ppm to about 50 ppm, and alternatively from about 25 ppm to about 50 ppm. The N-PKEK may be present in the compositions at from about 0.01 ppm to about 25 ppm, alternatively from about 0.05 ppm to about 20 ppm, from about 0.1 ppm to about 10 ppm, from about 0.2 ppm to about 10 ppm, from about 0.3 ppm to about 10 ppm, from about 0.5 ppm to about 10 ppm, from about 1 ppm to about 10 ppm, and alternatively from about 1 ppm to about 5 ppm.

### Dipeptide

The compositions herein include a safe and effective amount of the palmitoylated diopeptide, pal-KT (INCI: Palmitoyl Dipeptide-7). The pal-KT may be present in the present compositions at about 0.1 ppm to about 100 ppm, alternatively about 0.5 ppm to about 100 ppm, alternatively 1 ppm to about 100 ppm, alternatively about 1 to about 50 ppm, alternatively about 1 to about 25 ppm, alternatively about 1 to 10 ppm. The pal-KT may be present in the compositions at 3 ppm to 50 ppm, alternatively 5 ppm to about 30 ppm, alternatively from about 5 ppm to about 20 ppm, and alternatively 5 ppm to about 10 ppm. Pal-KT is available as Palestrina® from Sederma (France).

### Pentapeptide

The compositions herein include a safe and effective amount of the palmitoylated pentapeptide, pal-KTTKS [SEQ ID NO: 1] (INCI: Palmitoyl Pentapeptide-4). The pal-KTTKS may be present in the present compositions at about 0.1 ppm to about 100 ppm, alternatively about 0.5 ppm to about 100 ppm, alternatively 1 to about 100 ppm, alternatively about 1 to about 50 ppm, alternatively about 1 to about 25 ppm, alternatively about 1 to about 10 ppm. The pal-KTTKS may be present in the compositions at about 3 ppm to about 50 ppm, alternatively about 5 ppm to about 30 ppm, alternatively from about 5 ppm to about 20 ppm, and alternatively about 5 ppm to about 10 ppm. Pal-KTTKS is available as Promatrixyl® from Sederma® (France).

### Ratio of Peptides Attached to Fatty Acid Moieties to Tetrapeptide

The ratio of peptides that are attached to fatty acid moieties peptides to N-PKEK [SEQ ID NO: 1] can be present at a particular ratio to increase the skin penetration of N-PKEK [SEQ ID NO: 1]. The ratio of peptides that are attached to fatty acid moieties peptides to N-PKEK [SEQ ID NO: 1] can be from about 1:10 to about 3:1, alternatively from about 1.5:10 to about 2:1, alternatively from about 1:5 to about 1.5:1, alternatively from about 1:4 to about 1:1, alternatively from about 1:3 to about 1:1, alternatively from about 1:3 to about 2:3, and alternatively from about 1:3 to about 1:2. The ratio of peptides that are attached to fatty acid moieties peptides to N-PKEK [SEQ ID NO: 1] can be from about 1:20 to less than 1:1, alternatively from about 1:10 to about 1:2, alternatively from about 1.5:10 to about 2:5, and alternatively from about 2:5 to about 2.

### Vitamin B₃ Compound

The compositions herein can include a safe and effective amount of a vitamin B3 compound. The composition can contain from about 0.1% to about 10%, alternatively from about 0.5% to about 8%, alternatively from about 1% to about 7% of a vitamin B3 compound, alternatively from about 1% to about 6%, alternatively from about 2% to about 5%, alternatively from about 1% to about 5%, and alternatively from about 3% to about 4%. The vitamin B3 compound can be niacinamide.

As used herein, “vitamin B; compound” means a compound having the formula:

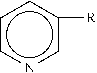

Where: R is CONH₂ (i.e., niacinamide), COOH (i.e., nicotinic acid) or CH₂OH (i.e., nicotinyl alcohol); derivatives thereof; and salts of any of the foregoing.

Exemplary derivatives of vitamin B3 compounds include nicotinic acid esters, including non-vasodilating esters of nicotinic acid (e.g., tocopheryl nicotinate, myristyl nicotinate) nicotinamide riboside, nicotinyl amino acids, nicotinyl alcohol esters of carboxylic acids, nicotinic acid N-oxide, and niacinamide N-oxide.

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. The carrier can be present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In some instances, the dermatologically acceptable carrier is in the form of an emulsion that has a continuous aqueous phase (e.g., an oil-in-water or water-in-oil-in-water emulsion) or a continuous oil phase (e.g., water-in-oil or oil-in-water-in-oil emulsion). The oil phase of the emulsion may include silicone oils, non-silicone oils such as hydrocarbon oils, esters, ethers, and mixtures thereof. The aqueous phase may include water and water-soluble ingredients (e.g., water-soluble moisturizing agents, conditioning agents, anti-microbials, humectants and/or other skin care actives). In some instances, the aqueous phase may include components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s).

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable diluents. As used herein, “diluent” refers to materials in which the skin care actives herein can be dispersed, dissolved, or otherwise incorporated. Some non-limiting examples of hydrophilic diluents include water, organic hydrophilic diluents such as lower monovalent alcohols (e.g., C₁-C₄) and low molecular weight glycols and polyols, including propylene glycol, polyethylene glycol (e.g., molecular weight of 200 to 600 g/mole), polypropylene glycol (e.g., molecular weight of 425 to 2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

### Conditioning Agents

The compositions herein may include 0.1% to 50% by weight of a conditioning agent (e.g., 0.5% to 30%, 1% to 20%, or even 2% to 15%). Adding a conditioning agent can help provide the composition with desirable feel properties (e.g., a silky, lubricious feel upon application). Some non-limiting examples of conditioning agents include, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof. Particularly suitable examples of conditioning agents include volatile or non-volatile silicone fluids such as dimethicone copolyol, dimethylpolysiloxane, diethylpolysiloxane, mixed C1-30 alkyl polysiloxanes, phenyl dimethicone, dimethiconol, dimethicone, dimethiconol, silicone crosspolymers, and combinations thereof. Dimethicone may be especially suitable, since some consumers associate the feel properties provided by certain dimethicone fluids with good moisturization. Other examples of silicone fluids that may be suitable for use as conditioning agents are described in U.S. Pat. No. 5,011,681.

### Rheology Modifiers

The compositions herein may include 0.1% to 5% of a rheology modifier (e.g., thickening agent) to provide the composition with suitable rheological and skin feels properties. Some non-limiting examples of thickening agents include crosslinked polyacrylate polymers, polyacrylamide polymers, polysaccharides, gums and mixtures thereof. In a particularly suitable example, the composition may include a superabsorbent polymer thickening agent such as sodium polyacrylate, starch grafted sodium polyacrylate, or a combination of these. Some non-limiting examples of superabsorbent polymer thickeners are described in, for example, U.S. Pat. No. 9,795,552.

Some consumers find compositions that use silicone fluids as conditioning agents to be undesirably greasy or heavy feeling. Thus, it may be desirable to provide a composition that is free of or substantially free of silicone fluid. It may also be desirable to tailor a superabsorbent polymer thickener to provide the composition with a light, airy feel, for example, by adjusting the amount of water in the composition, the water:oil ratio (e.g., 12:1 to 1:1), and/or the ratio of water to thickener or oil to thickener.

### Emulsifiers

When the dermatologically acceptable carrier is in the form of an emulsion, it may be desirable to include an emulsifier to provide a stable composition (e.g., does not phase separate). When included, the emulsifier may be present at an amount of 0.1% to 10% (e.g., 1% to 5%, or 2%-4%). Emulsifiers may be nonionic, anionic or cationic. Some non-limiting examples of emulsifiers that may be suitable for use herein are disclosed in U.S. Pat. Nos. 3,755,560; 4,421,769; and Mccutcheon's Detergents and Emulsifiers, North American Edition, pages 317-324 (1986).

### Other Optional Ingredients

The present composition may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

### Method of Use

The present method includes identifying a target portion of skin where treatment is desired and applying a composition comprising an effective amount of N-PKEK [SEQ ID NO: 1], one or more fatty acid moieties, such as palmitoyl including palmitoyl dipeptide-7 (pal-KT) and/or palmitoyl pentapeptide-4 (pal-KTTKS) [SEQ ID NO: 2], and optionally one or more additional skin care actives, to the target portion of skin. The target portion of skin may be on a facial skin surface such as the forehead, perioral, chin, periorbital, nose, and/or cheek) or another part of the body (e.g., hands, arms, legs, back, chest, underarms, underboobs, feet). The person or target portion of skin in need of treatment may be one that exhibits one or more hyperpigmented spots, uneven tone, and/or skin dullness. In some instances, a target portion of skin may not exhibit one or more hyperpigmented spots, uneven tone, and/or skin dullness, but a user may still wish to treat the portion of skin if it is one that commonly exposed to higher levels of exogenous stressors (e.g., sun exposed skin such as facial skin and arm skin). In this way, the present methods and compositions may be used prophylactically to help delay skin aging including hyperpigmented spots, uneven tone, and/or skin dullness.

The composition may be applied to a target portion of skin and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. The treatment period herein is ideally of sufficient time for the N-PKEK [SEQ ID NO: 1] and other peptides or actives, if present, to improve the appearance of the skin. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a hyperpigmented spot or portion thereof) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

### Definitions

“Apply” or “application,” as used in reference to a composition or material herein, means to place or spread the composition onto a human skin surface such as the epidermis.

“Derivative” means an acid, amide, ether, ester, amino, carboxyl, acetyl, alcohol derivative, or salts thereof of a given compound.

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein.

“Improve the appearance of” means providing a measurable, desirable change or benefit in skin appearance, which may be quantified, for example, by a decrease in redness, inflammation, and/or plaque scales.

“Skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin; preventing damage to skin via antioxidant approaches, including UV A and UV B induced damage, preventing formation of comedomes, balancing the skin microbiome or preventing acne.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Skin cell” refers to the types of cells commonly found in human skin. Non-limiting examples of skin cells are keratinocytes, fibroblasts, melanocytes, Langerhans cells, and Merkel cells.

As used herein, “treatment period,” means the length of time and/or frequency that a material or composition is applied to a target skin surface.

Other than in the operating examples, or where otherwise indicated, all numbers expressing quantities of ingredients and/or reaction conditions are to be understood as being modified in all instances by the term “about” which can encompass ±10%, ±8%, ±6%, ±5%, ±4%, ±3%, ±2%, ±1%, or ±0.5%.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. Unless otherwise indicated, all measurements are understood to be made at approximately 21° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

It is to be understood that, as used herein the terms “the,” “a,” or “an,” mean “at least one,” and should not be limited to “only one” unless explicitly indicated to the contrary.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

### Test Methods

### Skin Penetration Test Method

The skin penetration of N-PKEK was assessed using the Franz diffusion cell assay (T. J. Franz, J. Invest. Dermatol. 64:190-195, 1975; T. J. Franz, et al., Skin Pharmacol. Physiol. 22:276-286, 2009). The ³H-N-PKEK and optionally one or more additional peptides were formulated in a skin care composition. The formulations were spiked with ³H-N-PKEK. Six hours after topically dosed on human cadaver skin, residual product was removed from the skin surface, the epidermal and dermal layers were separated, and receptor solutions (transdermal) were collected. The penetration of ³H-N-PKEK through the skin was measured using liquid scintillation counting. The amount of ³H-N-PKEK that was present in the epidermis, dermis, and receptor was measured and the % of the ³H-N-PKEK present in each portion was calculated. It was assumed that the proportion of ³H-N-PKEK in each portion would be the same as the proportion of N-PKEK. To determine total skin penetration, the percentages of N-PKEK found in the epidermis, dermis, and receptors were added together. The percentage of N-PKEK remaining on the skin's surface was calculated by subtracting the total penetration percentage from 100%.

### Examples

The following examples are provided to help illustrate the skincare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

Example 1 was made and Examples 2-13 could be made using standard methods for formulating skin care compositions, such as oil-in-water emulsions, familiar to those skilled in the art.

TABLE 2
Examples 1-4

| | Example | | | |
|---|---|---|---|---|
| | 1 | 2 | 3 | 4 |
| Phase A | | | | |
| Cetyl Alcohol 95% | 0.5 | 0.4 | 0.5 | 1.00 |
| Stearyl Alcohol 97% | 0.6 | 0.8 | 1.0 | 1.2 |
| Behenyl Alcohol | 0.40 | 0.6 | | |
| PEG 100 Stearate | 0.10 | 0.1. | 0.2 | 0.2 |
| Cetearyl Glucoside & | 0.10 | 0.2 | 0.2 | 0.4 |
| Cetearyl AL | | | | |
| Isopropyl Isostearate | 1.33 | | | |
| Isohexadecane | 3.00 | 3.0 | | |
| Caprylic/Capric Triglyceride | | 1.0 | 3.0 | 3.0 |
| Butyrospermum Parkii (Shea | | | 1.0 | 4.0 |
| Butter) | | | | |
| Phase B | | | | |
| Panthenol (D-Panthenol Liquid) | 0.50 | 1 | 1 | 0.5 |
| Water | QS | QS | QS | QS |
| Glycerin 99% | 8 | 8 | 5 | 2 |
| Syniorage ™ PW¹ | 0.01 | 0.1 | 1 | 3 |
| Niacinamide | 5 | 5 | 3.5 | 2 |
| Pentylene glycol | 3 | 3 | 2 | 0 |
| 1,3- Butylene Glycol | 1.2 | | | |
| Disodium EDTA | 0.1 | 0.05 | 0.1 | |
| Phase C | | | | |
| TiO2 75% Water, Polyacrylate | 0.2 | 0.1 | 0 | 0 |
| PF | | | | |
| Polyacrylamide & C13-14 | 2.5 | 2 | 2 | 3.5 |
| Isoparaffin | | | | |
| Dimethicone & Dimethiconol | 2 | 2 | 1 | 0 |
| Atpeptide ™² | 0.2 | 0.1 | 0.1 | 0 |
| Palestrina ®³ | | 0.015 | 0.15 | 1.5 |
| Promatrixyl ®⁴ | | 0.01 | 0.1 | 1.0 |
| Phenoxylethanol | 0.38 | 0.3 | 0. | |
| Hexanediol & Caprylyl Glycol | 0.80 | 0.8 | 0.8 | 0.6 |
| N-PKEK | 0.004 | 0.00001 | 0.0050 | 0.01 |

### INCI and Suppliers for Table 2:

1. Mannitol (and) Acetyl Tetrapeptide-11 from BASF®
2. Aqua (and) Butylene Glycol (and) Tripeptide-3 from Ashland™
3. Water, glycerin, decyl glucoside, lactic acid, benzyl alcohol, and palmitoyl dipeptide-7 (pal-KT), from Sederma®
4. Palmitoyl pentapeptide-4 (pal-KTTKS) from Croda

TABLE 3
Examples 5-13

| | 5 | 6 | 7 | 8 | 9 | 10 | 11 | 12 | 13 |
|---|---|---|---|---|---|---|---|---|---|
| Phase A | | | | | | | | | |
| Water | QS | QS | QS | QS | QS | QS | QS | QS | QS |
| Glycerol | 5 | 7 | 3 | 15 | 7 | 5 | 5 | 3 | 5 |
| Disodium EDTA | 0.1 | 0.05 | 0.1 | 0.1 | 0.05 | 0.05 | 0.05 | 0.05 | 0.1 |
| Phase B | | | | | | | | | |
| Dimethicone 5 cSt | — | — | — | — | — | — | — | 10 | 15 |
| Dimethicone and Dimethicone | — | — | — | — | — | — | — | 13 | 15 |
| Crosspolymer | | | | | | | | | |
| Laureth-4 | — | — | — | — | — | — | — | 0.25 | 0.35 |
| Polysorbate 20 | — | — | — | — | — | — | — | 0.15 | 0.25 |
| Tapioca Starch and | — | — | — | — | — | — | — | 2.5 | 3.5 |
| Polymethylsilsesquioxane | | | | | | | | | |
| Avobenzone | — | — | — | 3 | — | 3 | — | — | — |
| Homosalate | — | — | — | 15 | — | 10 | — | — | — |
| Octisalate | — | — | — | 5 | — | 5 | — | — | — |
| Octocrylene | — | — | — | 2.6 | — | 9 | — | — | — |
| Isopropyl Isostearate | 5 | 2.5 | 1 | — | — | — | — | — | — |
| Isohexadecane | 1 | 1.5 | 3 | — | — | — | — | — | — |
| Cetyl Alcohol | 0.25 | 0.5 | 0.32 | 0.4 | 0.4 | 0.3 | 0.5 | — | — |
| Tocopherol Acetate | | 0.5 | 0.25 | 1 | 0.25 | 0.25 | 0.25 | — | — |
| PEG-100 Stearate | 0.2 | 0.1 | 0.1 | 0.3 | 0.1 | 0.2 | 0.1 | — | — |
| Stearyl Alcohol | 0.5 | 1.5 | 0.4 | 0.6 | 0.5 | 0.4 | 0.6 | — | — |
| Behenyl Alcohol | 0.4 | 1 | 0.5 | 0.5 | 0.4 | 0.35 | 0.5 | — | — |
| Ethyl Paraben | 0.2 | 0.15 | 0.2 | 0.25 | — | — | — | — | — |
| Propyl Paraben | 0.1 | 0.15 | 0.1 | 0.15 | — | — | — | — | — |
| Polymethylsilsesquioxane | 1.25 | 2.5 | 1 | — | — | — | — | — | — |
| Phase C | | | | | | | | | |
| Titanium Dioxide | — | 0.5 | — | 0.25 | — | — | — | — | — |
| Tapioca Starch and | — | — | — | — | — | 12 | | — | — |
| Polymethylsilsesquioxane | | | | | | | | | |
| Vinyl Dimethicone/Methicone | 1.5 | | 1.5 | 3.5 | 5 | — | 7.5 | — | — |
| Silsesquioxane Crosspolymer | — | — | — | — | 1.5 | 1 | 1.5 | — | — |
| Sodium Polyacrylate Starch | | | | | | | | | |
| Hydroxyethyl acrylate/sodium | 2 | 1.5 | 2.5 | 2 | — | — | — | 1.25 | 2 |
| acryloyldimethyltaurate | | | | | | | | | |
| copolymer | | | | | | | | | |
| Phase D | | | | | | | | | |
| Water | 5 | 10 | 10 | 5 | 10 | 10 | 10 | 5 | 10 |
| Pal-KT | 0.001 | 1 | 0.5 | 0.1 | 0.05 | 0.025 | 0.01 | 0.005 | 0.0005 |
| Pal-KTTKS [SEQ. ID NO: 2] | 0.001 | 1 | 5 | 0.1 | 0.05 | 0.025 | 0.01 | 0.005 | 0.0005 |
| N-PKEK [SEQ. ID NO: 1] | 0.004 | 1 | 6.25 | 1 | 0.1 | 0.005 | 0.05 | 0.0005 | 0.005 |
| Niacinamide | 3.5 | — | 3.5 | — | 4 | 5 | — | — | 2 |
| Dexpanthenol | 0.5 | 0.5 | 0.5 | 1 | 1 | 1.5 | 0.25 | 1 | 0.5 |
| Phase E | | | | | | | | | |
| Benzyl alcohol | 0.25 | 0.4 | 0.25 | 0.5 | — | — | — | — | — |
| Hexanediol and Caprylyl | — | — | — | — | 0.7 | 0.8 | 0.7 | 0.7 | 1 |
| Glycol | | | | | | | | | |
| Phenoxyethanol | — | — | — | — | 0.3 | 0.4 | 0.5 | 0.2 | 0.25 |
| Dimethicone/dimethiconoll | 0.5 | 1 | 2 | 1 | 2 | 2 | 1 | 1.75 | 1 |

### Combinations:

A. A skin care composition comprising:
   a. a combination of peptides comprising nicotinoyl tetrapeptide-30 (N-PKEK) [SEQ ID NO: 1], derivatives thereof, or mixtures thereof and one or more peptides that are attached to a fatty acid moiety;
   b. a dermatologically acceptable carrier;
wherein the composition increases the total skin penetration and/or the epidermal skin penetration of the N-PKEK [SEQ ID NO: 1] according to the Skin Penetration Test Method.
B. The skin care composition according to Paragraph A, wherein the fatty acid moiety comprises a palmitoyl moiety.
C. The skin care composition according to Paragraph B, wherein the palmitoyl moiety is palmitoyl dipeptide-7 (pal-KT) and/or palmitoyl pentapeptide-4 (pal-KTTKS) [SEQ ID NO: 2].
D. A skin care composition comprising:
   a. a combination of peptides comprising:
      i. from about 1 ppm to about 50 ppm of a nicotinoyl tetrapeptide-30 (N-PKEK) [SEQ ID NO: 1], derivatives thereof, or mixtures thereof; and
      ii. one or more peptides having a palmitoyl moiety chosen from palmitoyl dipeptide-7 (pal-KT), palmitoyl pentapeptide-4 (pal-KTTKS) [SEQ ID NO: 2], or mixtures thereof;
   b. a dermatologically acceptable carrier;
wherein the combination increases the total skin penetration and/or the epidermal skin penetration of the N-PKEK [SEQ ID NO: 1] according to the Skin Penetration Test Method.
E. The skin care composition according to Paragraphs A-E, wherein the composition from about 5 ppm to about 50 ppm of the N-PKEK [SEQ ID NO: 1], preferably from about 10 ppm to about 50 ppm of the N-PKEK [SEQ ID NO: 1], and more preferably from about 25 ppm to about 50 ppm of the N-PKEK [SEQ ID NO: 1].
F. The skin care composition according to Paragraphs A-E, wherein the composition comprises from about 5 ppm to about 200 ppm of the one or more peptides having a palmitoyl moiety, preferably from about 5 ppm to about 150 ppm of the one or more peptides having a palmitoyl moiety, more preferably from about 7 ppm to about 100 ppm of the one or more peptides having a palmitoyl moiety, even preferably from about 8 ppm to about 50 ppm of the one or more peptides having a palmitoyl moiety, even more preferably from about 8 ppm to about 30 ppm of the one or more peptides having a palmitoyl moiety, even more preferably from about 10 ppm to about 30 ppm of the one or more peptides having a palmitoyl moiety, and even more preferably from about 15 ppm to about 25 ppm of the one or more peptides having a palmitoyl moiety.
G. The skin care composition according to Paragraphs A-F, wherein the composition comprises from about 1 to about 100 ppm pal-KT, preferably about 1 to about 50 ppm pal-KT, more preferably about 1 to about 25 ppm pal-KT, and even more preferably about 1 to about 10 ppm pal-KT.
H. The skin care composition according to Paragraphs D-G, wherein the composition comprises from about 1 to about 100 ppm pal-KTTKS [SEQ ID NO: 2], preferably about 1 to about 50 ppm pal-KTTKS [SEQ ID NO: 2], more preferably about 1 to about 25 ppm pal-KTTKS [SEQ ID NO: 2], and even more preferably about 1 to about 10 ppm pal-KTTKS [SEQ ID NO: 2].
I. The skin care composition according to Paragraphs A-H, wherein the composition comprises pal-KT and pal-KTTKS [SEQ ID NO: 2].
J. The skin care composition according to Paragraphs A-I, wherein the total skin penetration of the N-PKEK is ≥20, preferably ≥21, more preferably ≥23, even more preferably ≥25, even more preferably ≥27, even more preferably ≥29, even more preferably ≥30, and most preferably ≥31 according to the Skin Penetration Test Method.
K. The skin care composition according to Paragraphs A-J, wherein the total skin penetration has at least a 1.1-fold increase as compared to the control, preferably at least a 1.15-fold increase as compared to the control, more preferably at least a 1.2-fold increase as compared to the control, even more preferably at least a 1.3-fold increase as compared to the control, even more preferably at least a 1.4-fold increase as compared to the control, even more preferably at least a 1.5-fold increase as compared to the control, even more preferably at least a 1.6-fold increase as compared to the control, more preferably at least a 1.65-fold increase as compared to the control, and even more preferably alternatively at least a 1.7-fold increase as compared to the control according to the Skin Penetration Test Method.
L. The skin care composition according to Paragraphs A-K, wherein the epidermal skin penetration is ≥10, preferably ≥12, more preferably ≥13, even more preferably ≥15, even more preferably ≥16, and most preferably ≥18, according to the Skin Penetration Test Method.
M. The skin care composition according to Paragraphs A-L, wherein the epidermal skin penetration has a 1.2-fold increase as compared to the control, preferably at least a 1.25-fold increase as compared to the control, more preferably at least a 1.3-fold increase as compared to the control, more preferably at least a 1.35-fold increase as compared to the control, more preferably at least a 1.38-fold increase as compared to the control, more preferably at least a 1.5-fold increase as compared to the control, more preferably at least a 1.6-fold increase as compared to the control, and even more preferably at least a 1.8-fold increase as compared to the control, according to the Skin Penetration Test Method.
N. The skin care composition according to Paragraphs A-M, further comprising a safe and effective amount of vitamin B3 compound.
O. The skin care composition according to Paragraph N, wherein the vitamin B3 compound is chosen from niacinamide, nicotinic acid, nicotinyl alcohol, or mixtures thereof.
P. The skin care composition according to Paragraph O, wherein the vitamin B3 compound is niacinamide.
Q. The skin care composition according to Paragraphs A-O, wherein the ratio of peptides that are attached to fatty acid moieties peptides to N-PKEK [SEQ ID NO: 1] is from about 1:10 to about 3:1, preferably from about 1.5:10 to about 2:1, more preferably from about 1:5 to about 1.5:1, even more preferably from about 1:4 to about 1:1, even more preferably from about 1:3 to about 1:1, even more preferably from about 1:3 to about 2:3, and most preferably from about 1:3 to about 1:2.
R. The skin care composition according to Paragraphs A-P, wherein the N-PKEK [SEQ ID NO: 1] comprises N-PKEK-acid and/or N-PKEK-amide.
S. The skin care composition according to Paragraphs A-R, wherein the skin care composition comprises from about 50 wt. % to about 99 wt. % of the dermatologically acceptable carrier, preferably from about 60 wt. % to about 98 wt. % of the dermatologically acceptable carrier, more preferably from about 70 wt. % to about 98 wt. % of the dermatologically acceptable carrier, and even more preferably from about 80 wt. % to about 95 wt. % of the dermatologically acceptable carrier.
T. The skin care composition according to Paragraphs A-S, wherein the dermatologically acceptable carrier comprises water.
U. The skin care composition according to Paragraphs A-T, wherein the skin care composition is an oil-in-water emulsion.
V. The skin care composition according to Paragraphs A-U, wherein the skin care composition is a face wash, body wash, or leave-on skin care product.
W. The skin care composition according to Paragraph V, wherein the skin care composition is a leave-on skin care product, and the leave-on skin care product is a moisturizer or a treatment product.
X. A skin care composition for use in a method of treating skin, comprising:
   a. identifying a target portion of skin where treatment is desired;
   b. applying the composition according to Paragraphs A-W.
Y. The method according to Paragraph W, wherein the composition is applied at least once per day for at least 2 weeks, preferably at least 4 weeks, more preferably at least 8 weeks, and most preferably at least 12 weeks.
Z. The method according to Paragraph W-Y, wherein the composition brightens and/or evens the tone of the target portion of skin.
AA. Use of N-PKEK and palmitoyl moiety is palmitoyl dipeptide-7 (pal-KT) and/or palmitoyl pentapeptide-4 (pal-KTTKS) [SEQ ID NO: 2] in a skin care composition according to Paragraphs A-W, to brighten and/or even skin tone.
BB. Use of N-PKEK and palmitoyl moiety is palmitoyl dipeptide-7 (pal-KT) and/or palmitoyl pentapeptide-4 (pal-KTTKS) [SEQ ID NO: 2] in a skin care composition according to Paragraphs A-W, to fade one or more dark spots.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition comprising:
a. a combination of peptides comprising:
i. from about 1 ppm to about 50 ppm of a nicotinoyl tetrapeptide-30 (N-PKEK) [SEQ ID NO: 1], derivatives thereof, or mixtures thereof; and
ii. one or more peptides having a palmitoyl moiety chosen from palmitoyl dipeptide-7 (pal-KT) and/or palmitoyl pentapeptide-4 (pal-KTTKS) [SEQ ID NO: 2];
b. a dermatologically acceptable carrier;
wherein the combination increases the total skin penetration and/or the epidermal skin penetration of the N-PKEK [SEQ ID NO: 1].

2. The skin care composition of claim 1, wherein the composition comprises from about 10 ppm to about 50 ppm of the N-PKEK [SEQ ID NO: 1].

3. The skin care composition of claim 1, wherein the composition comprises from about 5 ppm to about 200 ppm of the one or more peptides having a palmitoyl moiety.

4. The skin care composition of claim 1, wherein the composition comprises about 1 to about 100 ppm pal-KT.

5. The skin care compositions of claim 1, wherein the composition comprises about 1 to about 100 ppm pal-KTTKS [SEQ ID NO: 2].

6. The skin care composition of claim 1, wherein the composition comprises pal-KT and pal-KTTKS [SEQ ID NO: 2].

7. The skin care composition of claim 1, wherein the total skin penetration of the N-PKEK is ≥20, according to the Skin Penetration Test Method.

8. The skin care composition of claim 1, wherein the total skin penetration has at least a 1.1-fold increase as compared to the control, according to the Skin Penetration Test Method.

9. The skin care composition of claim 1, wherein the epidermal skin penetration is ≥10, according to the Skin Penetration Test Method.

10. The skin care composition of claim 1, wherein the epidermal skin penetration has a 1.2-fold increase as compared to the control, according to the skin Penetration Test Method.

11. The skin care composition of claim 1, further comprising a vitamin B3 compound chosen from niacinamide, nicotinic acid, nicotinyl alcohol, or mixtures thereof.

12. A skin care composition comprising:
a. a combination of peptides comprising nicotinoyl tetrapeptide-30 (N-PKEK) [SEQ ID NO: 1], derivatives thereof, or mixtures thereof and one or more peptides that are attached to a fatty acid moiety;
b. a dermatologically acceptable carrier;
wherein the composition increases the total skin penetration and/or the epidermal skin penetration of the N-PKEK [SEQ ID NO: 1].

13. The skin care composition of claim 12, wherein the fatty acid moiety comprises a palmitoyl moiety.

14. The skin care composition of claim 13, wherein the palmitoyl moiety is palmitoyl dipeptide-7 (pal-KT) and/or palmitoyl pentapeptide-4 (pal-KTTKS) [SEQ ID NO: 2].

15. The skin care composition of claim 12, wherein the ratio of peptides that are attached to fatty acid moieties peptides to N-PKEK [SEQ ID NO: 1] is from about 1:5 to about 1.5:1.

16. The skin care composition according to claim 12, wherein the skin care composition is a face wash, body wash, or leave-on skin care product.

17. The skin care composition according to claim 12, wherein the skin care composition is a leave-on skin care product, and the leave-on skin care product is a moisturizer or a treatment product.

18. A method of cosmetically treating skin comprising:
a. identifying a target portion of skin where treatment is desired;
b. applying the composition according to claim 1.

19. The method of claim 18, wherein the target portion of skin comprises a dark spot and the composition fades the dark spot.

20. The method of claim 18, wherein the composition brightens and/or evens the tone of the target portion of skin.


SEQUENCE LISTING (2 sequences)

SEQ ID NO: 1 - protein, 4 aa, synthetic construct
PKEK

SEQ ID NO: 2 - protein, 5 aa, synthetic construct
KTTKS